(19) United States
(12) Patent Application Publication
(10) Pub. No.: US 2004/0152784 A1
(43) Pub. Date: Aug. 5, 2004

(54) Pharmaceutical composition and method for treatment of a urea cycle deficiency or sickle-cell anaemia

(75) Inventor: Graham Alan March, Surrey (GB)

Correspondence Address: SENNIGER POWERS LEAVITT AND ROEDEL, ONE METROPOLITAN SQUARE, 16TH FLOOR, ST LOUIS, MO, 63102, US

(73) Assignee: Special Products Limited

(21) Appl. No.: 10/622,891

(22) Filed: Jul. 17, 2003

Related U.S. Application Data
(60) Provisional application No. 60/397,828, filed on Jul. 23, 2002.

(51) Int. Cl. A61K 31/192; A61K 9/00

(52) U.S. Cl. 514/570; 424/400

(57) ABSTRACT

A pharmaceutical composition is disclosed comprising sodium 4-phenylbutyrate, an effective amount of at least one aromatic flavoring agent, and an effective amount of at least one synthetic sweetening agent. Also disclosed is a method of treatment of a urea cycle deficiency or sickle-cell anaemia.

### REFERENCE TO RELATED APPLICATIONS

This application claims priority from U.S. provisional application Serial No. 60/397,828, filed on Jul. 23, 2002.

### FIELD OF THE INVENTION

This invention relates to a pharmaceutical composition. In particular, it relates to a pharmaceutical composition suitable for oral administration for the treatment of urea cycle deficiencies. It further relates to a pharmaceutical composition suitable for oral administration for the treatment of sickle-cell anaemia. Methods are also disclosed for the treatment of urea cycle deficiencies as well as of sickle-cell anaemia.

### BACKGROUND TO THE INVENTION

Some children are born with a rare enzyme deficiency in the urea cycle. For example, a child may suffer from an N-acetylglutamine synthetase 1 deficiency or from an ornithine transcarbamoylase deficiency. Consequently, such a child is unable to excrete waste nitrogen as urea. The waste nitrogen accumulates as ammonium ions in the child's plasma. This results in a condition known as hyperammonaemia. The accumulation of ammonium ions in the child's plasma is first manifested as food aversion and nausea and, if the enzyme deficiency is severe, the condition leads to coma and death within a few days of birth.

At present the genetic defect cannot be cured, but the condition can be treated by adherence to a life-long low protein diet and the administration of suitable medication. If the child has an N-acetylglutamine synthetase 1 deficiency, then the treatment involves administration of sodium phenylacetate and sodium benzoate. If the child has an ornithine transcarbamoylase deficiency, then treatment involves administration of sodium 4-phenylbutyrate (typically in a dosage of 450-600 mg/kg/day in three or more divided doses). The sodium 4-phenylbutyrate is converted to 2-phenylacetate, which combines with the amino acid, glutamine, in the plasma and is excreted as phenylacetylglutamine in the urine. Thus, sodium 4-phenylbutyrate provides an alternative to urea as a means of excreting waste nitrogen from the body.

Sodium 4-phenylbutyrate is a very bitter compound and has a pungent odor of mice. This combination makes the compound very unacceptable to children who have to take large amounts of the medicine every day. A six year old child weighing 19 kg, for example, typically has to take 3.8 g of powder three times daily. This tends to lead to lack of compliance in taking the prescribed dose at the required intervals. Consequently, some children have to be admitted to hospital two or three times a year because they feel nauseous, this being a first sign of hyperammonaemia, and cannot take the powder orally. In hospital, the patient is treated with sodium 4-phenylbutyrate (or with sodium phenylacetate and sodium benzoate) administered as an intravenous infusion to reduce the ammonium ion level to normal. When the nausea subsides, normal oral therapy is then resumed. Sodium 4-phenylbutyrate injections (and also mixed sodium phenylacetate and sodium benzoate injections) are only stocked by specialist hospitals which have the equipment to monitor hyperammonaemia. Sometimes the delay in reaching such a hospital leads to the patient being admitted in a hyperammonaemic coma. Death may result or, on recovery, the child may be permanently brain-damaged.

At the present time, sodium 4-phenylbutyrate is licensed in the USA, where it is sold under the trade name BUPHENYL, and in Europe under the trademark AMMONAPS. It is marketed as a granular powder for young children and as 500 mg tablets for adults and children weighing over 20 kg. The marketing authorisation holder in Europe is Orphan Europe of Immeuble “Le Guillaumet”, F-92046, Paris La Défense, France. It is reported that AMMONAPS granules contain calcium stearate and colloidal anhydrous silica as excipients. Orphan Europe recommends that the powder is measured in one of three different sizes of measuring spoon. The recommended method of measuring a dose is to remove a heaped measuring spoon from the container and to draw a flat surface, such as a knife blade, across the top of the measure. However, the accuracy of dosage is imprecise since the bulk density of the granular powder can be variable and in many cases the dose for a young child cannot be measured exactly since the use of a measuring spoon may give a dose that is inappropriate having regard to the child's weight.

Another use for sodium phenylbutyrate is for the induction of foetal haemoglobin production in subjects with sickle cell anaemia; this has been described by George J. Dover (Blood, Vol. 84, No. 1, Jul. 1, 1994: pp 339-343). This paper notes that a major drawback to the use of sodium phenylbutyrate is the high dose. It goes on to say:

“[Sodium phenylbutyrate] in powder form has a bitter taste that, despite many attempts, cannot be disguised. Two of the three subjects treated after discharge from the hospital reported inability to maintain compliance”.

### BRIEF SUMMARY OF THE INVENTION

There is accordingly a need to provide an improved pharmaceutical composition containing sodium 4-phenylbutyrate for use in the treatment of patients suffering from urea cycle deficiencies. There is a further need for a pharmacologically acceptable form of sodium 4-phenylbutyrate which improves patient compliance and minimises the need for frequent hospitalisation. There is also a need for an improved form of sodium 4-phenylbutyrate suitable for the treatment of sickle-cell anaemia. In addition, there is a need for an improved method of treating urea cycle deficiencies and also a similar need for an improved method of treating sickle-cell anaemia.

The present invention accordingly seeks to provide a preparation capable of delivering the requisite dose of sodium 4-phenylbutyrate in a form which is readily acceptable to young patients and measurable with suitable accuracy. It also seeks to provide improved methods for treating not only urea cycle deficiencies but also sickle-cell anaemia.

According to a first aspect of the present invention, there is provided a pharmaceutical composition comprising sodium 4-phenylbutyrate, an effective amount of at least one aromatic flavoring agent, and an effective amount of at least one synthetic sweetening agent.

Also provided in accordance with the invention is a dry powder pharmaceutical composition comprising sodium 4-phenylbutyrate, an effective amount of at least one water soluble sweetening agent, and an effective amount of at least one water soluble flavoring agent, the effective amounts being selected so as to mask substantially the bitter taste and pungent odor of sodium 4-phenylbutyrate.

In another aspect the invention provides a concentrated aqueous solution containing at least about 200 mg/ml of sodium 4-phenylbutyrate up the solubility limit thereof measured at 10° C., and having dissolved therein an effective amount of at least one water soluble sweetening agent, and an effective amount of at least one water soluble flavoring agent, the effective amounts being selected so as to mask substantially, following dilution by at least about 5 fold up to about 10 fold or more with water, the bitter taste and pungent odor of sodium 4-phenylbutyrate.

In another aspect of the present invention there is provided a unit dose for administration to a patient requiring treatment for a urea cycle deficiency according to a regime in which the patient is administered a predetermined number of doses daily corresponding to from about 450 to about 600 mg/kg/day of sodium 4-phenylbutyrate, the unit dose prepared by diluting with water an aliquot of a concentrated aqueous solution containing at least about 200 mg/ml of sodium 4-phenylbutyrate up the solubility limit thereof measured at 10° C., an effective amount of at least one water soluble sweetening agent, and an effective amount of at least one water soluble flavoring agent, the unit dose containing from about 10 to about 50 mg/ml, typically about 25 mg/ml, of sodium 4-phenylbutyrate and the effective amounts being selected so as to mask substantially the bitter taste and pungent odor of sodium 4-phenylbutyrate.

In a still further aspect of the present invention there is provided a pharmaceutically acceptable aqueous solution ready for administration to a patient requiring treatment for a urea cycle deficiency according to a regime in which the patient is administered a predetermined number of doses daily corresponding to from about 450 to about 600 mg/kg/day of sodium 4-phenylbutyrate, the solution containing a unit dose of sodium 4-phenylbutyrate, an amount of at least one water soluble sweetening agent, and an amount of at least one water soluble flavouring agent, the concentration of sodium 4-phenylbutyrate in the aqueous solution ranging from about 10 to about 50 mg/ml and the amounts of the at least one water soluble sweetening agent and of the at least one water soluble flavoring agent being selected so as to mask substantially the bitter taste and pungent odor of sodium 4-phenylbutyrate.

In addition the invention provides a pharmaceutical composition comprising granules comprising sodium 4-phenylbutyrate and a binding amount of a binding agent, the composition further including an effective amount of at least one synthetic water soluble sweetening agent, and an effective amount of at least one water soluble flavoring agent, the amounts of the at least one artificial water soluble sweetening agent and of the at least one water soluble flavoring agent being sufficient, upon dissolution in water to yield an aqueous solution containing from about 10 to about 50 mg/ml of sodium 4-phenylbutyrate, to render the composition palatable to a child.

There is also provided in accordance with the present invention in a method of treating a patient suffering from a condition selected from a urea cycle deficiency and sickle-cell anaemia which comprises administering to the patient in one or more unit doses daily a pharmaceutical composition comprising sodium 4-phenylbutyrate in an amount corresponding to from about 450 to about 600 mg/kg/day, the improvement comprising administering sodium 4-phenylbutyrate in the form of an aqueous solution comprising sodium 4-phenylbutyrate, an effective amount of at least one water soluble sweetening agent, and an effective amount of at least one water soluble fruit flavoring agent, the effective amounts being selected so as to mask substantially the bitter taste and pungent odor of sodium 4-phenylbutyrate.

According to a still further aspect of the present invention there is provided a method of manufacturing a pharmaceutical composition comprising sodium 4-phenylbutyrate, the method comprising:

(i) providing a solution of a binding agent in a volatile solvent therefor;

(ii) admixing a predetermined volume of the solution of the binding agent with a predetermined quantity of sodium 4-phenylbutyrate to form a wetted mass;

(iii) forming the wetted mass into granules; and

(iv) drying the granules to remove essentially all of the volatile solvent therefrom and form dry granules; and

further including the step of incorporating into the composition an effective amount of at least one water soluble sweetening agent and an effective amount of at least one water soluble flavoring agent to form a pharmaceutical composition;

wherein the effective amounts are selected so that, upon dissolution of the pharmaceutical composition in water to form a solution containing from about 10 to about 50 mg/ml of sodium 4-phenylbutyrate, the bitter taste and pungent odor of sodium 4-phenylbutyrate is effectively masked.

### BRIEF DESCRIPTION OF THE DRAWING

FIG. 1 is a schematic representation of the mode of action of sodium benzoate and sodium 4-phenylbutyrate in the treatment of hyperammonaemia.

### DETAILED DESCRIPTION OF THE INVENTION

The invention provides a pharmaceutical composition comprising sodium 4-phenylbutyrate, an effective amount of at least one aromatic flavoring agent, and an effective amount of at least one synthetic sweetening agent.

In such a pharmaceutical composition the flavoring agent is desirably a flavoring agent that is attractive to children. Thus the flavoring agent may be selected from fruit flavoring agents, preferably water soluble fruit flavoring agents, such as blackcurrant flavoring agent, cranberry flavoring agent, red cherry flavoring agent, black cherry flavoring agent, orange flavoring agent, lemon flavoring agent, raspberry flavoring agent, mango flavoring agent, banana flavoring agent, and strawberry flavoring agent. The preferred flavoring agent is a strawberry flavoring agent. Alternatively a non-fruit flavoring agent may be used.

The synthetic sweetening agent preferably comprises at least one synthetic sweetening agent selected from aspartame and potassium acesulfame. In a preferred composition the synthetic sweetening agent comprises a mixture of aspartame and potassium acesulfame.

The invention also includes within its scope a dry powder pharmaceutical composition comprising sodium 4-phenylbutyrate, an effective amount of at least with at least one water soluble sweetening agent, and an effective amount of at least one water soluble flavoring agent. The effective amounts in such a dry powder composition are selected so as to mask substantially the bitter taste and pungent odor of sodium 4-phenylbutyrate.

Preferably the pharmaceutical composition comprises granules comprising sodium 4-phenylbutyrate and an effective amount of a binding agent, such as polyvinylpyrrolidone (or povidone, as it is frequently termed). Preferably the binding agent is Povidone B.P. (The abbreviation “B.P.” stands for British Pharmacopeia).

A preferred pharmaceutical composition according to the invention comprises per 100 parts by dry weight of the composition:

from about 80 to about 90 parts by weight (preferably from about 82.5 to about 88.5 parts by weight) of sodium 4-phenylbutyrate;

from about 2.5 to about 5.0 parts by weight (preferably about 3.25 to about 4.5 parts by weight) of aspartame;

from about 1.5 to about 3.5 parts by weight (preferably about 1.75 to about 3.25 parts by weight) of potassium acesulfame;

from about 2.5 to about 5.0 parts by weight (preferably about 3.25 to about 4.5 parts by weight) of a fruit flavouring agent; and

from about 3.5 to about 6.5 parts by weight (preferably about 3.25 to about 5.25 parts by weight) of a binding agent.

The invention also provides a concentrated aqueous solution containing at least about 200 mg/ml of sodium 4-phenylbutyrate up the solubility limit thereof measured at 10° C., and having dissolved therein an effective amount of at least one water soluble sweetening agent, and an effective amount of at least one water soluble flavoring agent. The effective amounts in this case are preferably selected so as to mask substantially, following dilution by at least about 5 fold up to about 10 fold or more with water, the bitter taste and pungent odor of sodium 4-phenylbutyrate.

The invention also encompasses in a method of treating a patient suffering from a condition selected from a urea cycle deficiency and sickle-cell anaemia which comprises administering to the patient in one or more unit doses daily a pharmaceutical composition comprising sodium 4-phenylbutyrate in an amount corresponding to from about 450 to about 600 mg/kg/day, the improvement comprising administering sodium 4-phenylbutyrate in the form of an aqueous solution comprising sodium 4-phenylbutyrate, an effective amount of at least one water soluble sweetening agent, and an effective amount of at least one water soluble fruit flavoring agent. The effective amounts are desirably selected so as to mask substantially the bitter taste and pungent odor of sodium 4-phenylbutyrate, while the flavoring agent can be, for example, a strawberry flavoring agent. Moreover the synthetic sweetening agent desirably comprises at least one synthetic sweetening agent selected from aspartame and potassium acesulfame, for example a mixture of aspartame and potassium acesulfame.

A unit dose for a child patient requiring treatment can be prepared by diluting with water an aliquot of such a concentrated aqueous solution. In such a treatment there may be used a regime in which the patient is administered a predetermined number of unit doses daily corresponding to from about 450 to about 600 mg/kg/day of sodium 4-phenylbutyrate. The predetermined number of unit doses may range from 1 to 5 or more, but conveniently the patient is given 3 unit doses per day. Typically the aliquot of the concentrated aqueous solution is diluted with water to form a unit dose until the concentration of sodium 4-phenylbutyrate is from about 10 to about 50 mg/ml, for example about 25 mg/ml. The amount of sodium 4-phenylbutyrate in the unit dose will depend upon the number of doses that the patient's daily regime demands. Since the patient is typically required to take three divided doses per day, typically the amount of sodium 4-phenylbutyrate in the unit dose corresponds to about one third of the recommended daily requirement of about 450 to 600 mg/kg/day. The unit dose thus comprises sodium 4-phenylbutyrate, an effective amount of at least one water soluble sweetening agent, and an effective amount of at least one water soluble flavoring agent, the effective amounts of the at least one sweetening agent and of the at least one water soluble sweetening agent being selected so as to mask substantially the bitter taste and pungent odor of sodium 4-phenylbutyrate.

Spray dried sodium 4-phenylbutyrate typically has a bulk density of about 26.4 g/100 ml (± about 15%). Hence a 25 g quantity of sodium 4-phenylbutyrate would fill or almost fill a 100 ml bottle. It is also somewhat hygroscopic. Its flow properties mean that the spray dried material does not flow well from a hopper into a bottle during a filling process in a factory.

In order to overcome these problems it is preferred to granulate sodium 4-phenylbutyrate in the course of manufacturing a pharmaceutical preparation according to the invention. Hence a pharmaceutical composition can be prepared which comprises granules comprising sodium 4-phenylbutyrate, and a binding agent, such as polyvinylpyrrolidone, the composition further comprising an effective amount of at least one synthetic water soluble sweetening agent, and an effective amount of at least one water soluble flavoring agent. In such a composition the amounts of at least one artificial water soluble sweetening agent and of the at least one water soluble flavoring agent should be sufficient, upon dissolution in water to yield a solution containing about 25 mg/ml of sodium 4-phenylbutyrate, to render the resulting aqueous solution palatable to a child despite the unpleasant taste and odor of sodium 4-phenylbutyrate.

To make such a pharmaceutical composition, one method comprises providing a solution of a binding agent in a volatile solvent therefor, followed by admixing a predetermined volume of the solution of the binding agent with a predetermined quantity of sodium 4-phenylbutyrate to form a wetted mass, and then forming the wetted mass into granules, which are then dried to remove essentially all of the volatile solvent therefrom and form dry granules. The resulting dry granules are then mixed with an effective amount of at least one water soluble sweetening agent and with an effective amount of at least one water soluble flavoring agent to form a pharmaceutical composition. The effective amounts are selected so that, upon dissolution of the pharmaceutical composition in water to form a solution containing from about 10 to about 50 mg/ml of sodium 4-phenylbutyrate, the bitter taste and pungent odor of sodium 4-phenylbutyrate is effectively masked.

In a variant of this method the predetermined volume of the solution of the binding agent is mixed with the predetermined quantity of sodium 4-phenylbutyrate, with the effective amount of the at least one synthetic water soluble sweetening agent, and with the at least one water soluble flavoring agent to form the wetted mass.

A pharmaceutical composition made by either of such methods typically has a bulk density of about 52.6 g/100 ml. Moreover it flows well from a hopper and hence is suitable for use in a bottle filling step in a factory environment. A 25 g quantity of the granulated pharmaceutical composition will thus approximately half fill a 100 ml bottle.

The binding agent can be any suitable pharmaceutically acceptable binding agent, such as polyvinylpyrrolidone. Preferably the solvent used for dissolution of the binding agent is non-toxic and has a boiling point of less than about 100° C., preferably less than about 80° C. As suitable solvents there can be mentioned alcohols, such as ethyl alcohol or iso-propyl alcohol, ketones, such as acetone, or esters, such as ethyl acetate.

Drying is preferably carried out by heating the wetted granules in an oven at an elevated temperature but below the boiling point of the solvent, for example at a temperature of from about 40° C. to about 60° C., and preferably no more than about 50° C., when the solvent is iso-propyl alcohol.

In a preferred aspect of the invention, the preparation is sold in the form of a water soluble dry powder or granulate composition. This is preferably dissolved to or near to saturation in pure water by a pharmacist before being dispensed to the consumer. The concentrate is preferably diluted according to taste by the patient (or the patient's guardian/carer) immediately prior to consumption. It is envisaged that the final dilute solution will contain the required dose of sodium 4-phenylbutyrate. Since the preparation is supplied to the consumer as a liquid concentrate, it is preferred that a graduated syringe is also supplied. This will enable the exact amount of concentrate to be measured by the patient, or by the patient's guardian or carer, according to individual needs. Typically, the prescribed dose as measured by the syringe is diluted before ingestion. Thus, for example, 1 part of the concentrated solution can be diluted with about 9 parts water, according to taste, immediately before use to form a long drink. Thus 15.2 ml of the concentrated solution containing 3.8 g of sodium 4-phenylbutyrate can, for example, be diluted to 150 ml with water.

The flavoring agent and the sweetening agent are preferably provided at the minimum level necessary to mask the flavor and odor of the sodium 4-phenylbutyrate. The maximum amounts of these components will be further limited by their solubilities and acceptable daily intakes.

It is intended that the pharmaceutical composition should be sold in dry form for dissolution by a pharmacist in purified water to form a concentrated aqueous solution containing, for example, from about 200 mg/ml of sodium 4-phenylbutyrate up to the solubility limit thereof in water at about 10° C. Normally such a concentrated solution contains no more than about 250 mg/ml of sodium 4-phenylbutyrate. It is important that the sweetening agent or agents and the flavoring agent should be fully dissolved in this concentrated aqueous solution. Hence the amounts of the sweetening agent or agents and of the flavoring agent should be selected in relation to the quantity of sodium 4-phenylbutyrate so that, at the chosen concentration of sodium 4-phenylbutyrate in the concentrated aqueous solution, the solubility limits of the sweetening agent or agent and of the flavoring agent are not exceeded at room temperature or lower, e.g. at about 10° C.

The long term consumption of excessive quantities of artificial sweeteners can be toxic. Therefore, each sweetener has an Acceptable Daily Intake (ADI), expressed in terms of mg/kg body weight per day, that has been shown to be safe. The properties of some available sweeteners are shown in Table 1. Aspartame is the sweetener with the most acceptable taste and, fortunately, has a high ADI. Unfortunately, it has a low solubility in water (1% w/v) and in aqueous solution it hydrolyses to phenylalanine within a few weeks and loses its sweetness. To minimize hydrolysis and the subsequent loss of sweetness, the pharmaceutical composition is desirably presented in accordance with the invention as a dry powder in a medicine bottle (e.g. 28.7 g in a 100 or 125 ml medicine bottle) with a child-resistant closure. To this dry powder the pharmacist then adds a suitable volume of purified water (e.g. 80 ml purified water to make 100 ml of solution in the case of a 100 or 125 ml medicine bottle) when the product is dispensed. The date of re-constitution is written on the label by the pharmacist and the label desirably advises the patient to discard any residual medicine 28 days after dispensing.

The perception of sweetness produced by aspartame is sustained, but is less rapid in onset than that produced by sugar. Therefore, potassium acesulfame is used in a preferred embodiment of the invention in combination with aspartame. Potassium acesulfame is desirably not used as the primary sweetener since it is sickly sweet to many people and the sensation of sweetness is not well sustained. Moreover it has an ADI of no more than 15 mg. However, it tends to generate a rapid onset in the perception of sweetness. Thus a small proportion of potassium acesulfame (i.e. an amount which will not exceed its ADI) in combination with aspartame generates a sensation of sweetness that is rapid in onset and sustained in action. These features are extremely desirable when consuming a few hundred milliliters of what would, otherwise, be a very bitter solution.

The properties of various artificial sweeteners are summarised in Table 1.

TABLE 1

| | Acceptable | Sweetness | | |
|---|---|---|---|---|
| | Daily Intake | Relative | Solubility | |
| Sweetener | (mg/kg/day) | to Sucrose | in Water | Taste |
| Potassium | 15 mg | × 200 | Very soluble | Sickly sweet |
| acesulfame | | | | Rapid onset |
| | | | | but not |
| | | | | sustained |
| Aspartame | 50 mg | × 200 | Max. 1% w/v | Good |
| | | | (10 mg in 1 ml) | Slow onset |
| | | | Also | but sustained |
| | | | hydrolyses | |
| | | | significantly to | |
| | | | phenylalanine | |
| | | | (non-sweet) | |
| | | | within | |
| | | | 6 months | |
| Maltitol | None | × 1 | Very soluble | Good |
| (“Lycasin”) | | | | |
| Sodium | 5 mg | × 30 | Soluble | Good |
| cyclamate | | | | |
| Sodium | 5 mg | × 300 | Very soluble | Good but |
| Saccharine | | | | bitter after-taste |

Note:
Aspartame is the most acceptable sweetener, but its use is limited in liquids by its low solubility (maximum 1% w/v in liquids) and fairly rapid hydrolysis in water.

Table 2 illustrates the intake of various artificial sweeteners, expressed in mg/kg/day, at the maximum dose of 600 mg/kg/day of sodium 4-phenylbutyrate.

TABLE 2

| | | | Volume of sodium 4- | |
|---|---|---|---|---|
| | Acceptable | Content of sweetener | phenylbutyrate solution | |
| | Daily | in a 250 mg/ml | (250 mg/ml) | Amount of sweetener |
| | Intake | solution of sodium | required to deliver a | consumed per kg |
| Sweetener | mg/kg/day | 4-phenylbutyrate | 600 mg dose | per day |
| Acesulfame K | 15 mg | 6 mg/ml | 2.4 ml | 14.4 mg |
| Fast dissolution | | | | (6 mg × 2.4) |
| Stable in water | | | | |
| Aspartame | 50 mg | 10 mg/ml | 2.4 ml | 24 mg (10 mg × 2.4) |
| Slow onset | | (max 1% w/v soluble) | | |
| Long duration | | i.e. 10 mg in 1 ml | | |
| Max 1% solubility in water | | | | |
| Hydrolyses in water | | | | |
| Sodium cyclamate | 5 mg | — | — | — |
| Maltitol powder | Similar to | — | — | — |
| | sugar | | | |
| Sodium saccharin | — | — | — | — |
| Not allowed in USA | | | | |

The medicine is reconstituted by dissolution in purified water by the pharmacist to form a concentrated aqueous solution which is at a concentration close to the maximum solubility of sodium 4-phenylbutyrate (250 mg/ml) at 10° C. This will prevent the active ingredient precipitating if the bottle is inadvertently stored in a cold place. A compact 100 ml or 125 ml bottle can contain 2.2 days' supply for a child consuming 15.2 ml of the concentrated aqueous solution three times daily. It has been found that a concentration of about 200 mg/ml sodium 4-phenylbutyrate is also the maximum that can be sweetened by using aspartame as the sole sweetening agent.

Only 24 mg/kg/day of the ADI of aspartame (maximum 50 mg/kg/day) is used since its use is limited by its low solubility (10 mg/ml).

Potassium acesulfame is preferably used at a concentration in the concentrated aqueous solution which is just below the limit of its ADI, i.e 14.4 mg of the maximum 15 mg ADI (see Table 2).

Table 3 summarises the intake of artificial sweeteners at the maximum dose of 600 mg/kg/day of sodium 4-phenylbutyrate.

TABLE 3

| | | Content of sweetener | Volume of sodium 4- | Amount of |
|---|---|---|---|---|
| | Acceptable | suggested to sweeten | phenylbutyrate solution | sweetener consumed |
| | Daily | a 150 mg in 1 ml | (150 mg/ml) required to | per kg |
| | Intake | solution of sodium | deliver a 600 mg/kg dose | body weight per |
| Sweetener | mg/kg/day | 4-phenylbutyrate | of sodium 4-phenylbutyrate | day |
| Acesulfame K | 15 mg | 3.75 mg/ml | 4 ml | 15 mg |
| 200 × sucrose | | | | (3.75 mg × 4) |
| Fast dissolution | | | | |
| Stable in water | | | | |
| Aspartame | 50 mg | 10 mg/ml | 4 ml | 40 mg |
| 200 × sucrose | | (max 1% w/v soluble) | | (10 mg × 4) |
| Slow onset | | i.e. 10 mg in 1 ml | | |
| Long duration | | | | |
| Max 1% solubility in water | | | | |
| Hydrolyses in water | | | | |

It has been found that it is unnecessary to incorporate a preservative in the formulation to prevent bacterial and fungal growth. Thus it has been demonstrated that sodium 4-phenylbutyrate prevents such growth in the concentrated flavored solution at a concentration of 250 mg/ml. This may not apply at much lower concentrations. Therefore, the concentrated solution should be diluted by the patient's carer or guardian immediately before it is consumed by the patient. Thus the patient's carer or guardian should not prepare a jug of ready-to-use liquid to last a week or so.

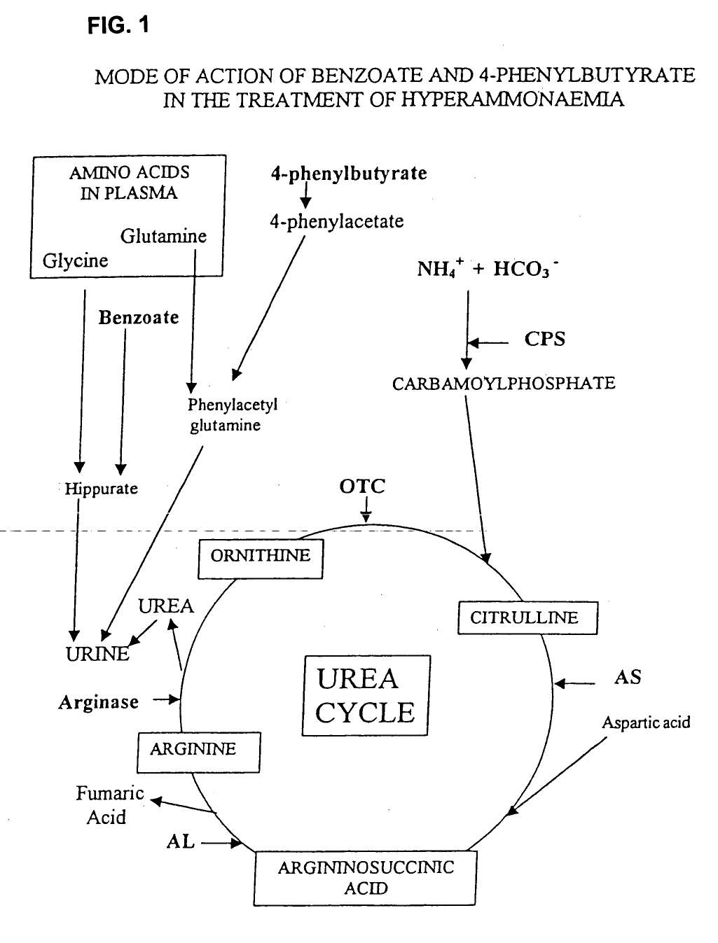

FIG. 1 illustrates diagrammatically metabolic pathways by which the human body excretes the nitrogen content of amino acids present in plasma. Because of the participation of α-ketoglutarate in numerous transaminations, glutamate is a prominent intermediate in nitrogen elimination as well as in anabolic pathways. Glutamate formed in the course of nitrogen elimination is either oxidatively deaminated by glutamate dehydrogenase in the liver, thereby forming ammonia, or converted to glutamine by glutamine synthase and transported to kidney tubule cells. There the glutamine is sequentially deamidated by glutaminase and deaminated by glutamate dehydrogenase in the kidney. The ammonia produced in the latter two reactions is excreted, along with urea, as NH₄⁺ ions in the urine, which help to maintain urine pH in the normal range of pH 4 to pH 8. Normal serum ammonium concentrations are in the range of 20 to 40 mM and an increase to about 400 mM causes alkalosis and neurotoxicity.

Ammonium ions react in the presence of carbamoyl phosphate synthetase-1 (“CPS”) to form carbamoyl phosphate which condenses with ornithine in the presence of ornithine transcarbamoylase (“OTC”) to produce citrulline. The energy for this reaction is provided by the high energy anhydride of carbamoyl phosphate. The product, citrulline, is converted by reaction with aspartic acid, catalysed by arginosynthetase (“AS”), to argininosuccinic acid. Arginine and fumarate are produced from argininosuccinic acid by the enzyme argininosuccinate lysase. In the final stage of the cycle arginase cleaves aspartate to form ornithine and urea. The urea can then be excreted in urine.

If a child is born with a deficiency in one of the enzymes of the urea cycle, such as N-acetylglutamine synthetase 1, then the child may suffer from symptoms such as ataxia, convulsions, lethargy, poor feeding and eventually coma and death, if not recognised and treated properly. Such a urea cycle defect is known as Type 1 hyperammonaemia which is conventionally treated by administration of sodium benzoate which binds glycine covalently and forms hippurate. On the other hand children having an ornithine transcarbamoylase deficiency are said to suffer from Type 2 hyperammonaemia which is normally treated by administration of sodium 4-phenylbutyrate. Sodium 4-phenylbutyrate is metabolised to sodium phenylacetate, which in turn reacts with glutamine to form phenylacetylglutamine. The resulting phenylacetylglutamine is mainly excreted in the child's urine, although a minor amount may also be excreted in the child's faeces.

The invention is further illustrated by way of the following Examples.

### EXAMPLE 1

The following ingredients were used to form a dry granulated powder composition:

| Component | Weight | |
|---|---|---|
| Sodium 4-phenylbutyrate | 12.1 | kg |
| Aspartame | 484 | g |
| Strawberry flavor powder | 484 | g |
| Potassium acesulfame | 193 | g |
| Povidone B.P. | 639 | g |

| | |
|---|---|
| Iso-propyl alcohol | q.s. |

The Povidone B.P. was dissolved in the minimum volume of isopropyl alcohol using a high shear mixer. (The amount of isopropyl alcohol needed is typically no more than about 2 l). The resulting solution was mixed with the sodium 4-phenylbutyrate to form a dough. This dough was forced through a sieve (British Standard 22 mesh, i.e. 0.710 mm) to form granules which were then spread on stainless steel trays and placed in an oven which was heated to about 50° C. until essentially all of the iso-propyl alcohol had been evaporated. The resulting “cake” was again passed through a sieve into a stainless steel drum to which the remaining dry ingredients were then added. The drum was then tumbled to blend the powder mixture until it was homogeneously mixed.

### EXAMPLE 2

28.7 g of the granulated product of Example 1 was introduced under clean conditions into a 125 ml amber glass bottle which was then sealed and labelled. The label bore the appropriate information laid down under current United States legislation, including the name of the active ingredient, batch number, expiry date of the dry powder, and instructions for reconstitution as a concentrated liquid by a pharmacist, instructions to put the date of reconstitution on the label, instructions for further dilution by the patient's carer or guardian, and a warning to prepare each dilution immediately before consumption. In addition the label included advice to the patient to discard any residual medicine 28 days after dispensing. The bottle was then packaged together with a graduated syringe capable of withdrawing from the bottle a measured dose of the concentrated liquid, as well as a leaflet with instructions for the parent or guardian responsible for administering the pharmaceutical composition to a child patient suffering from hyperammonaemia or from sickle cell anaemia.

### EXAMPLE 3

At the time of dispensing a pharmacist opened the bottle of Example 2 and added 80 ml of purified water to dissolve the charge of dry powder granulate. In addition he added the date upon which he had effected dissolution of the granulate to form a concentrated liquid. Since the concentrated liquid had been reconstituted close to, but below, the maximum solubility of sodium 4-phenylbutyrate at 10° C., i.e. 250 mg/ml, the active ingredient did not precipitate even if the bottle was inadvertently stored in a cold place.

### EXAMPLE 4

The quantity of concentrated syrup liquid in Example 3 was sufficient to provide 2.2 days' supply for a child patient weighing 19 kg, based upon a dose of 15.2 ml of concentrated liquid taken 3 times daily. Prior to the patient taking a unit dose, its parent or guardian was instructed to withdraw a 15.2 ml aliquot of concentrated liquid from the bottle using the syringe provided and to squirt this quantity of concentrated liquid into a beaker or other drinking vessel followed by dilution of the concentrated liquid to approximately 150 ml using tap water or another form of potable water, before ingestion by the patient.

### EXAMPLE 5

In an alternative process for preparing a dry granulated pharmaceutical composition according to the invention the same ingredients are used as in Example 1 and in the same quantities. The difference between the process described in Example 1 and this alternative process is that the solution of Povidone B.P. is mixed with all of the other ingredients before the resulting dough is forced through a 12 mesh (1.70 mm) mesh sieve and then drying is effected at a temperature of less than 50° C. so as to avoid loss of the volatile aromatic oils of the strawberry flavor.

1. A pharmaceutical composition comprising sodium 4-phenylbutyrate, an effective amount of at least one aromatic flavoring agent, and an effective amount of at least one synthetic sweetening agent.

2. A pharmaceutical composition according to claim 1, in which the aromatic flavoring agent is selected from fruit flavoring agents.

3. A pharmaceutical composition according to claim 2, in which the aromatic flavoring agent is a strawberry flavoring agent.

4. A pharmaceutical composition according to claim 1, in which the synthetic sweetening agent comprises at least one synthetic sweetening agent selected from aspartame and potassium acesulfame.

5. A pharmaceutical composition according to claim 1, in which the synthetic sweetening agent comprises a mixture of aspartame and potassium acesulfame.

6. A pharmaceutical composition according to claim 1, in which the sodium 4-phenylbutyrate is present in the form of granules which further comprise an effective amount of a binding agent.

7. A pharmaceutical composition according to claim 6, which comprises, per 100 parts by dry weight of the composition;
from about 80 to about 90 parts by weight of sodium 4-phenylbutyrate;
from about 3.5 to about 5.0 parts by weight of aspartame;
from about 1.5 to about 3.5 parts by weight of potassium acesulfame;
from about 2.5 to about 5.0 parts by weight of an aromatic fruit flavoring agent; and
from about 3.5 to about 6.5 parts by weight of a binding agent.

8. A pharmaceutical composition according to claim 7, in which the binding agent is polyvinylpyrrolidone.

9. A pharmaceutical composition according to claim 7, in which the fruit flavoring agent is a strawberry flavoring agent.

10. A dry powder pharmaceutical composition comprising sodium 4-phenylbutyrate, an effective amount of at least one water soluble sweetening agent, and an effective amount of at least one water soluble flavoring agent, the effective amounts being selected so as to mask substantially the bitter taste and pungent odor of sodium 4-phenylbutyrate.

11. A pharmaceutical composition which comprises granules comprising sodium 4-phenylbutyrate, a binding agent, an effective amount of at least one synthetic water soluble sweetening agent, and an effective amount of at least one water soluble flavoring agent, the effective amounts being selected so that, upon dissolution in water to yield an aqueous solution that contains from about 10 to about 50 mg/ml of sodium 4-phenylbutyrate, the resulting aqueous solution is palatable.

12. A pharmaceutical composition according to claim 11, which comprises per 100 parts by weight of the composition;
from about 80 to about 90 parts by weight of sodium 4-phenylbutyrate;
from about 3.5 to about 5.0 parts by weight of aspartame;
from about 1.5 to about 3.5 parts by weight of potassium acesulfame;
from about 2.5 to about 5.0 parts by weight of a strawberry flavoring agent; and
from about 3.5 to about 6.5 parts by weight of polyvinylpyrrolidone.

13. A concentrated aqueous solution containing at least about 200 mg/ml of sodium 4-phenylbutyrate up the solubility limit thereof measured at 10° C., and having dissolved therein an effective amount of at least one water soluble sweetening agent, and an effective amount of at least one water soluble flavoring agent, the effective amounts being selected so as to mask substantially, following dilution by at least about 5 fold up to about 10 fold or more with water, the bitter taste and pungent odor of sodium 4-phenylbutyrate.

14. A concentrated aqueous solution according to claim 13, in which the flavoring agent is selected from fruit flavoring agents.

15. A concentrated aqueous solution according to claim 13, in which the flavoring agent is a strawberry flavoring agent.

16. A concentrated aqueous solution according to claim 13, in which the synthetic sweetening agent comprises at least one synthetic sweetening agent selected from aspartame and potassium acesulfame.

17. A concentrated aqueous solution according to claim 13, in which the synthetic sweetening agent comprises a mixture of aspartame and potassium acesulfame.

18. A concentrated aqueous solution according to claim 13, which comprises, per 100 parts by weight of the dry components of the composition;
from about 80 to about 90 parts by weight of sodium 4-phenylbutyrate;
from about 3.5 to about 5.0 parts by weight of aspartame;
from about 1.5 to about 3.5 parts by weight of potassium acesulfame;
from about 2.5 to about 5.0 parts by weight of a fruit flavoring agent; and
from about 3.5 to about 6.5 parts by weight of polyvinylpyrrolidone.

19. A concentrated aqueous solution according to claim 17, in which the fruit flavoring agent is a strawberry flavoring agent.

20. A unit dose for administration to a patient requiring treatment for a urea cycle deficiency according to a regime in which the patient is administered a predetermined number of doses daily corresponding to from about 450 to about 600 mg/kg/day of sodium 4-phenylbutyrate, the unit dose prepared by diluting with water an aliquot of a concentrated aqueous solution containing at least about 200 mg/ml of sodium 4-phenylbutyrate up the solubility limit thereof measured at 10° C., an effective amount of at least one water soluble sweetening agent, and an effective amount of at least one water soluble flavoring agent, the unit dose containing from about 10 to about 50 mg/ml of sodium 4-phenylbutyrate and the effective amounts being selected so as to mask substantially the bitter taste and pungent odor of sodium 4-phenylbutyrate.

21. A unit dose according to claim 20, in which the amount of sodium 4-phenylbutyrate corresponds to no more than about one third of the maximum daily requirement of about 600 mg/kg/day.

22. A pharmaceutically acceptable aqueous solution ready for administration to a patient requiring treatment for a urea cycle deficiency according to a regime in which the patient is administered a predetermined number of doses daily corresponding to from about 450 to about 600 mg/kg/day of sodium 4-phenylbutyrate, the solution containing a unit dose of sodium 4-phenylbutyrate, an amount of at least one water soluble sweetening agent, and an amount of at least one water soluble flavoring agent, the concentration of sodium 4-phenylbutyrate in the aqueous solution ranging from about 10 to about 50 mg/ml and the amounts of the at least one water soluble sweetening agent and of the at least one water soluble flavoring agent being selected so as to mask substantially the bitter taste and pungent odor of sodium 4-phenylbutyrate.

23. A pharmaceutical composition comprising granules comprising sodium 4-phenylbutyrate and a binding amount of a binding agent, the composition further including an effective amount of at least one synthetic water soluble sweetening agent, and an effective amount of the at least one water soluble flavoring agent, the amounts of at least one artificial water soluble sweetening agent and of the at least one water soluble flavoring agent being sufficient upon dissolution in water to yield an aqueous solution containing from about 10 to about 50 mg/ml of sodium 4-phenylbutyrate to render the resulting aqueous solution palatable to a child.

24. A pharmaceutical composition according to claim 23, in which the binding agent comprises polyvinylpyyrolidone.

25. A pharmaceutical composition according to claim 23, in which the flavoring agent is selected from fruit flavoring agents.

26. A pharmaceutical composition according to claim 25, in which the flavoring agent is a strawberry flavoring agent.

27. A pharmaceutical composition according to claim 23, in which the synthetic sweetening agent comprises at least one synthetic sweetening agent selected from aspartame and potassium acesulfame.

28. A pharmaceutical composition according to claim 23, in which the synthetic sweetening agent comprises a mixture of aspartame and potassium acesulfame.

29. A pharmaceutical composition according to claim 23, which comprises, per 100 parts by dry weight of the composition;
from about 82.5 to about 99.5 parts by weight of sodium 4-phenylbutyrate;
from about 3.25 to about 4.5 parts by weight of aspartame;
from about 1.75 to about 3.25 parts by weight of potassium acesulfame;
from about 3.25 to about 4.5 parts by weight of a water soluble fruit flavoring agent; and
from about 3.25 to about 5.25 parts by weight of polyvinylpyrrolidone.

30. A pharmaceutical composition according to claim 29, in which the fruit flavoring agent is a strawberry flavoring agent.

31. A pharmaceutical composition according to claim 23, wherein the granules comprise sodium 4-phenylbutyrate and the binding agent and wherein the granules are mixed with the at least one synthetic water soluble softening agent and with the at least one water soluble flavoring agent to form the wetted mass.

32. A pharmaceutical composition according to claim 23, wherein the granules comprise sodium 4-phenylbutyrate, the binding agent, the at least one synthetic water soluble sweetening agent and the at least one water soluble flavoring agent.

33. In a method of treating a patient suffering from a condition selected from a urea cycle deficiency and sickle-cell anaemia which comprises administering to the patient in one or more unit doses daily a pharmaceutical composition comprising sodium 4-phenylbutyrate in an amount corresponding to from about 450 to about 600 mg/kg/day, the improvement comprising administering sodium 4-phenylbutyrate in the form of an aqueous solution comprising sodium 4-phenylbutyrate, an effective amount of at least one water soluble sweetening agent, and an effective amount of at least one water soluble fruit flavoring agent, the effective amounts being selected so as to mask substantially the bitter taste and pungent odor of sodium 4-phenylbutyrate.

34. A method according to claim 33, in which the fruit flavoring agent is a strawberry flavoring agent.

35. A method according to claim 33, in which the synthetic sweetening agent comprises at least one synthetic sweetening agent selected from aspartame and potassium acesulfame.

36. A method according to claim 33 in which the synthetic sweetening agent comprises a mixture of aspartame and potassium acesulfame.

37. A method according to claim 36, in which the amounts of aspartame and potassium acesulfame are selected so as not to exceed their respective Acceptable Daily Intakes.

38. A method according to claim 33, in which the composition comprises, per 100 parts by dry weight of the composition:
from about 80 to about 90 parts by weight of sodium 4-phenylbutyrate;
from about 3.5 to about 5.0 parts by weight of aspartame;
from about 1.5 to about 3.5 parts by weight of potassium acesulfame;
from about 2.5 to about 5.0 parts by weight of a fruit flavoring agent; and
from about 3.5 to about 6.5 parts by weight of a binding agent.

39. A method according to claim 38, in which the fruit flavoring agent is a strawberry flavoring agent.

40. A method according to claim 33, in which the unit dose is prepared by diluting with water a concentrated aqueous solution containing at least about 200 mg/ml of sodium 4-phenylbutyrate up the solubility limit thereof measured at 10° C., an effective amount of at least one water soluble sweetening agent, and an effective amount of at least one water soluble flavoring agent, the effective amounts being selected so as to mask substantially, following dilution by from about 5 times up to about 10 times or more with water, the bitter taste and pungent odor of sodium 4-phenylbutyrate.

41. A method according to claim 40, in which the at least one water soluble flavoring agent is selected from fruit flavoring agents.

42. A method according to claim 41, in which the at least one water soluble flavoring agent comprises a strawberry flavoring agent.

43. A method according to claim 40, in which the synthetic sweetening agent comprises at least one synthetic sweetening agent selected from aspartame and potassium acesulfame.

44. A method according to claim 40, in which the synthetic sweetening agent comprises a mixture of aspartame and potassium acesulfame.

45. A method according to claim 42, in which the concentrated aqueous solution comprises, per 100 parts by dry weight of the components;
from about 82.5 to about 88.5 parts by weight of sodium 4-phenylbutyrate;
from about 3.25 to about 4.5 parts by weight of aspartame;
from about 1.75 to about 3.25 parts by weight of potassium acesulfame;
from about 3.25 to about 4.5 parts by weight of an aromatic fruit flavoring agent; and
from about 3.25 to 5.25 parts by weight of a binding agent.

46. A method according to claim 45, in which the binding agent comprises polyvinylpyrrolidone.

47. A method according to claim 33, in which the patient is administered three unit doses daily and in which the amount of sodium 4-phenylbutyrate in the unit dose corresponds to no more than about one third of the maximum daily requirement of about 600 mg/kg/day.

48. A method according to claim 45, in which the condition is a urea cycle deficiency.

49. A method according to claim 45, in which the condition is sickle-cell anaemia.

50. A method of manufacturing a pharmaceutical composition comprising sodium 4-phenylbutyrate, the method comprising:
(i) providing a solution of a binding agent in a volatile solvent therefor;
(ii) admixing a predetermined volume of the solution of the binding agent with a predetermined quantity of sodium 4-phenylbutyrate to form a wetted mass;
(iii) forming the wetted mass into granules;
(iv) drying the granules to remove essentially all of the volatile solvent therefrom and form dry granules; and
further including the step of incorporating into the composition an effective amount of at least one synthetic water soluble sweetening agent and an effective amount of at least one water soluble flavoring agent to form a pharmaceutical composition;
wherein the effective amounts are selected so that, upon dissolution of the pharmaceutical composition in water to form a solution containing from about 10 to about 50 mg/ml of sodium 4-phenylbutyrate, the bitter taste and pungent odor of sodium 4-phenylbutyrate is effectively masked.

51. A method according to claim 50, wherein the admixing step (ii) comprises admixing the predetermined volume of the solution of the binding agent with the predetermined quantity of sodium 4-phenylbutyrate, with the effective amount of the at least one synthetic water soluble sweetening agent, and with the effective amount of the at least one water soluble flavoring agent to form the wetted mass.

52. A method according to claim 50, wherein the dry granules of step (iv) are mixed with the effective amount of the at least one synthetic water soluble sweetening agent and with the effective amount of the water soluble flavoring agent.

53. A method according to claim 50, in which the water soluble flavoring agent is a fruit flavoring agent.

54. A method according to claim 50, in which the water soluble flavoring agent is a strawberry flavoring agent.

55. A method according to claim 50, in which the synthetic sweetening agent comprises at least one synthetic sweetening agent selected from aspartame and potassium acesulfame.

56. A method according to claim 50, in which the synthetic sweetening agent comprises a mixture of aspartame and potassium acesulfame.

57. A method according to claim 50, in which the composition comprises, per 100 parts by dry weight of the composition:
from about 80 to about 90 parts by weight of sodium 4-phenylbutyrate;
from about 3.5 to about 5.0 parts by weight of aspartame;
from about 1.5 to about 3.5 parts by weight of potassium acesulfame;
from about 2.5 to about 5.0 parts by weight of a fruit flavoring agent; and
from about 3.5 to about 6.5 parts by weight of a binding agent.

58. A method according to claim 57, in which the fruit flavoring agent is a strawberry flavoring agent.

59. A method according to claim 50, in which the volatile solvent is iso-propyl alcohol.

60. A method according to claim 59, in which the granules are dried at a temperature of from about 40 to about 60° C.

61. A method according to claim 60, in which the granules are dried at a temperature of about 50° C.

62. A method according to claim 50, in which the binding agent is polyvinylpyrrolidone.
